(19) United States
(12) Patent Application Publication
(10) Pub. No.: US 2006/0052684 A1
(43) Pub. Date: Mar. 9, 2006

(54) Medical cockpit system

(76) Inventors: Takashi Takahashi, Hyogo (JP); Hiroshi Oyama, Hyogo (JP); Tomohiro Kuroda, Osaka (JP); Kenta Hori, Osaka (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 10/512,076

(22) PCT Filed: May 7, 2003
(86) PCT No.: PCT/JP03/05729

§ 371 Date: Nov. 5, 2004

(30) Foreign Application Priority Data
May 7, 2002 (JP) 2002-131944

(51) Int. Cl. A61B 5/05

(52) U.S. Cl. 600/407

(57) ABSTRACT

Measurement information of a patient 11 in a clinic, video of a medical procedure site, voice in the clinic, and video information of a body including an affected part of the patient are integrated and sent to a medical cockpit through a network 40. In the medical cockpit, the video of the clinic is outputted on a large screen monitor 73, and the sent measurement information and a plurality of sets of body video information are switched and inset in predetermined positions in the monitor 73 and reproduced. An enlarged moving image of the affected part is reproduced on a main monitor 71. Measurement information of the patient 11, moving image information such as video information of the body including the affected part of the patient or enlarged image information is reproduced on an auxiliary monitor 72. The voice information of the clinic is reproduced by a surround speaker 76. A doctor operates a medical procedure section 62 and controls a knife operating section 121 of a robot 12 in the clinic while seeing the images on the main monitor and the auxiliary monitor.

### TECHNICAL FIELD

The present invention relates to a medical cockpit system for carrying out medical procedure such as examination, inspection, diagnosis, treatment and other proceeding by remote control, and relates to a medical cockpit used for the medical cockpit system.

### BACKGROUND TECHNIQUE

In recent years, as information network technique is progressing, remote surgical operation in which a plurality of medical locations are connected to each other and surgical operation of a patient who is at a remote location is carried out by remote control is in the limelight. Japanese Patent Application Laid-open No. H7-184923 discloses an apparatus which carried out such remote surgical operation. According to a method described in this publication, surgical operation is carried out by remote control in a narrow space. The apparatus includes operation environment information detecting means which detects images of an affected part and its periphery and a contact force of a tip end of a working machine to the affected part. This apparatus drives operation tools in accordance with motions of a doctor who performs the surgical operation (simply “doctor”, hereinafter) based on information obtained by processing the former information, and in accordance with a command value generated by the contact force of the tip end of the working machine to the affected part.

According to this apparatus, the images of the affected part and its periphery and the contact force information of the tip end of the working machine to the affected part are transmitted to the doctor, but information in the operation room required by the doctor is not always transmitted, and sufficient information is not transmitted in various remote surgical operations in some cases.

In a network type surgical operation, since the doctor and the patient do not exist in the same site, the doctor carries out the surgical operation by operating a robot from a remote location. In this case, the doctor must concentrate on the operation of the surgical site, and must obtain various information sent from the operation staff and measuring instruments in an operation room at the same time. To make it possible to do this, it is necessary to appropriately dispose, around the operator, not only the surgical site, but also video information, voice information and the like around the medical robot periphery and a second participant medical location which are related to the surgical site.

It is an object of the present invention to provide an environment where a doctor who participates in a network type medical care, especially surgical operation from a remote location can smoothly access information and can appropriately receive necessary notifications.

That is, it is an object of the invention to provide a medical cockpit system in which a clinic and a doctor who performs the surgical operation existing at a remote location are connected to each other, all information of clinic required by a doctor is transmitted to the remote location, and space where it looks as if the doctor existing at the remote location is directly carrying out the medical procedure is created. It is another object of the invention to provide a medical cockpit used for the medical cockpit system, and a clinic for realizing this medical cockpit system.

It is another object of the invention to make it possible to provide information using peripheral vision or stereophonic by using an immersive display and a multi-speaker system so that a doctor can smoothly access the information.

### DISCLOSURE OF THE INVENTION

A first aspect of the present invention provides a medical cockpit system in which a clinic and a medical cockpit are connected to each other through a network, the clinic comprises measurement information obtaining means for obtaining measurement information such as electrocardiogram information of a patient, clinic camera means for shooting a state in the clinic including at least a medical procedure table, clinic voice information obtaining means for obtaining voice in the clinic, physical information camera means for shooting a body including an affected part of the patient, and medical procedure means for carrying out medical procedure for the patient by remote control, and the medical cockpit comprises monitor means for outputting video from the measurement information obtaining means, the clinic camera means and the physical information camera means, voice reproducing means for reproducing voice information from the clinic voice information obtaining means, and an operating section for remote controlling the medical procedure means, wherein the monitor means comprises first monitor means disposed at a predetermined distance from an operator who operates the operating section, and second monitor means disposed around hands of the operator, the first monitor means outputs a peripheral vision video in the clinic shot by the clinic video means and outputs a plurality of local videos in the clinic as catalog screens such that the local videos are partially overlapped on the peripheral vision video, and the second monitor means outputs measurement information obtained by the measurement information obtaining means or video from the physical information camera means.

According to this aspect, the operator can concentrate on the operation of a surgical site using the second monitor means, grasp the entire states of the peripheral vision in the clinic using the first monitor means, and grasp the details of especially required video as local video. Therefore, in this aspect, the operator can grasp the peripheral information in the clinic required by the operator. Therefore, it is possible to create space in which it looks as if the operator existing at a remote location exists in the clinic, and the invention can provide an environment where the operator directly carries out medical procedure at the remote location.

According to a second aspect of the invention, in the medical cockpit system of the first aspect, the medical cockpit system further comprises database means in which previously obtained physical data of the patient or data peculiar to the patient is accumulated, wherein data accumulated in the database means is sent together with the information in the clinic.

According to this aspect, even if a patient exists at a remote location, the medical procedure can be carried out at a remote location while seeing, using the monitor means, data concerning the patient required for the medical procedure.

According to a third aspect of the invention, in the medical cockpit system of the first aspect, the medical cockpit system further comprises sending means which sends the information sent from the clinic not only to the medical cockpit but also to another cockpit.

According to this aspect, a diagnosis doctor or other operator who does not exist in the clinic carries out appropriate diagnosis in accordance with the proceedings of the medical procedure and in such a state, the doctor or the operator can give advice to the clinic and another operator or can take part in the surgical operation. Further, the medical cockpit system can be utilized as educational or internship site for an intern.

According to a fourth aspect of the invention, in the medical cockpit system of the first aspect, the physical information camera means comprises a plurality of camera apparatuses which shoot the affected part from a plurality of directions.

According to this aspect, an operator who is at a remote location can observe a portion receiving the medical procedure from multidirectional aspects, and can precisely grasp a position relation between an affected part and the operator.

According to a fifth aspect of the invention, in the medical cockpit system of the first aspect, the first monitor means is disposed such that a viewing angle in the horizontal direction at a position of the operator who operates the operating section is in a range of 120° to 330°.

According to this aspect, video information closer to peripheral information obtained at medical site can be obtained. If the first monitor means is disposed in a range of 270° or greater, it is preferable because peripheral vision angle of a person can be covered.

According to a sixth aspect of the invention, in the medical cockpit system of the first aspect, the second monitor means comprises a main monitor and an auxiliary monitor, the main monitor outputs the measurement information obtained by the measurement information obtaining means or the video from the physical information camera means, and the auxiliary monitor selectively switches and outputs catalog screens outputted on the first monitor means.

According to this aspect, the operator can carry out medical procedure while seeing another required video near his or her hand in a state in which the operator concentrates on the monitor means near the hands.

According to a seventh aspect of the invention, in the medical cockpit system of the first aspect, the second monitor means comprises a plurality of monitors, at least one of the monitors does not switch videos during operation of the medical procedure system.

According to this aspect, it is possible to completely avoid a case in which an operator erroneously switches video which he or she should not taking eyes off.

According to an eighth aspect of the invention, in the medical cockpit system of the sixth or seventh aspect, the auxiliary monitor switches the catalog screens by detecting voice, motion and countenance of the operator.

According to this aspect, the operator can display necessary information near his or her hand without moving the hand off the knife.

According to a ninth aspect of the invention, in the medical cockpit system of the sixth or seventh aspect, the auxiliary monitor switches the catalog screens using a foot switch.

According to this aspect, the operator can display necessary information near his or her hand without moving the hand off the knife.

According to a tenth aspect of the invention, in the medical cockpit system of the first aspect, the medical cockpit comprises cockpit camera means for shooting motion or countenance of the operator, and cockpit voice information obtaining means for obtaining voice of the operator, video shot by the cockpit camera means and voice obtained by the cockpit voice information obtaining means are sent to the clinic through the network.

According to this aspect, it is possible to grasp the state of the operator even in the clinic, total communication including gesture and hand gesture of the operator can be realized, and it is possible to carry out smooth communication swiftly.

According to an eleventh aspect of the invention, in the medical cockpit system of the first aspect, the clinic voice information obtaining means is a microphone held by a staff in the clinic.

According to this aspect, since an operator who is at a remote location can know voce of a staff in the clinic in real time, it is possible to further precisely know the state of the clinic, and to give appropriate instructions to the staff.

According to a twelfth aspect of the invention, in the medical cockpit system of the first aspect, the clinic voice information obtaining means is a microphone which captures sound in the vicinity of the affected part of the patient and breathing sound of the patient.

According to this aspect, an operator who is at a remote location can precisely determine the proceeding state of the medical procedure and an abnormal condition of a patient.

According to a thirteenth aspect of the invention, in the medical cockpit system of the first aspect, the catalog screen in the first monitor means is disposed in a video position on which video contents outputted on the catalog screen is outputted, of videos outputted by the peripheral vision video.

According to this aspect, since the respective local videos are disposed in the peripheral vision video in a corresponding manner, it is possible to precisely determine the necessary local video.

According to a fourteenth aspect of the invention, in the medical cockpit system of the first aspect, the local video outputted on the catalog screen of the first monitor means is video information which is intermittently received.

According to this aspect, since it is not always necessary that the number of sending lines is the same as the number of camera means, more local videos can easily be handled, and a larger amount of video information required for the operator can be provided.

According to a fifteenth aspect of the invention, in the medical cockpit system of the first aspect, the voice reproducing means is a multi-speaker system, the voice is reproduced in a stereophonic manner such that the voice can be heard at a position of the operator in the medical cockpit in the same direction or at the same distance as that of the voice heard at an original position of the operator in the clinic.

According to this aspect, since the voice in the clinic can be reproduced with precise localization and with enhanced ambience, it is possible to hear the voice as if the operator exists in the clinic.

According to a sixteenth aspect of the invention, in the medical cockpit system of the first aspect, the voice reproducing means is a multi-speaker system, when voice is generated from a peripheral vision video outputted by the monitor means or from video outputted by the local video, the voice is reproduced in a stereophonic manner so that the voice can be heard in correspondence with the position of the video.

According to this aspect, when the operator hears abnormal sound, he or she can see necessary video information immediately.

According to a seventeenth aspect of the invention, in the medical cockpit system of the second aspect, data stored in the database means is superposed on video of the physical information camera means in a transparent manner, or displayed adjacent to the video of the physical information camera means.

According to this aspect, it is possible to carry out appropriate medical procedure while confirming an affected part, e.g., tomography image such as MRI.

An eighteenth aspect of the invention provides a clinic connected to a medical cockpit through a network, comprising measurement information obtaining means for obtaining measurement information such as electrocardiogram information of a patient, clinic camera means for shooting a state in the clinic including at least a medical procedure table, clinic voice information obtaining means for obtaining voice in the clinic, physical information camera means for shooting a body including an affected part of the patient, and medical procedure means for carrying out medical procedure for the patient by remote control, and a monitor for reproducing video and voice of the operator, wherein information from the measurement information obtaining means, the clinic camera means, the clinic voice information obtaining means and the physical information camera means is sent to the medical cockpit, video and voice information of the operator from the medical cockpit is received, and the medical procedure means is allowed to be operated based on the information from the medical cockpit.

According to this aspect, when medical procedure is carried out at a remote location, it is possible to precisely transmit all necessary information of a clinic to an operator who is at a location away from the clinic. It is possible to grasp the state of the operator even in the clinic, total communication including gesture and hand gesture of the operator can be realized, and it is possible to carry out smooth communication swiftly.

A nineteenth aspect of the invention provides a medical cockpit connected to a clinic through a network, comprising monitor means for outputting video of the clinic, voice reproducing means for reproducing voice in the clinic, an operating section for remote controlling medical procedure means of the clinic, cockpit camera means for shooting motion and countenance of an operator, and cockpit voice information obtaining means for obtaining voice of the operator, wherein video outputted by the monitor means and voice reproduced by the voice reproducing means are received from the clinic, and information from the operating section, the cockpit camera means and the cockpit voice information obtaining means is sent to the clinic.

According to this aspect, it is possible to create space in which it looks as if the operator existing at a remote location away from the clinic exists in the clinic, it is possible to grasp the state of the operator even in the clinic, total communication including gesture and hand gesture of the operator can be realized, and it is possible to realize an environment in which the operator can directly carry out medical procedure at a remote location.

According to a twentieth aspect of the invention, in the medical cockpit of the nineteenth aspect, the monitor means comprises first monitor means disposed at a predetermined distance from an operator who operates the operating section, and second monitor means disposed around hands of the operator, the first monitor means outputs a peripheral vision video in the clinic shot by the clinic video means and outputs a plurality of local videos in the clinic as catalog screens such that the local videos are partially overlapped on the peripheral vision video.

According to this aspect, the operator can grasp the entire state of the peripheral vision in the clinic using the first monitor means, and grasp the details of especially required video as local video. Therefore, in this aspect, the operator can grasp the peripheral information in the clinic required by the operator. Therefore, it is possible to create space in which it looks as if the operator existing at a remote location exists in the clinic, and the invention can provide an environment where the operator directly carries out medical procedure at the remote location.

According to a twenty first aspect of the invention, in the medical cockpit of the twentieth aspect, the catalog screen in the first monitor means is disposed in a video position on which video contents outputted on the catalog screen is outputted, of videos outputted by the peripheral vision video.

According to this aspect, since the respective local videos are disposed in the peripheral vision video in a corresponding manner, it is possible to instantaneously grasp the position in the peripheral vision video, and to precisely determine the necessary local video.

According to a twenty second aspect of the invention, in the medical cockpit of the twentieth aspect, the second monitor means comprises a main monitor and an auxiliary monitor, the main monitor outputs measurement information obtained by the measurement information obtaining means or video from the physical information camera means.

According to this aspect, the operator can concentrate on the operation of a surgical site using the main monitor.

According to a twenty third aspect of the invention, in the medical cockpit of the twentieth aspect, the second monitor means comprises a main monitor and an auxiliary monitor, the auxiliary monitor selectively switches and outputs catalog screens outputted on the first monitor means.

According to this aspect, the operator can concentrate on the operation of a surgical site using the main monitor.

According to a twenty fourth aspect of the invention, in the medical cockpit of the nineteenth aspect, the medical cockpit is connected to a plurality of clinics, the monitor means comprises first monitor means disposed at a predetermined distance from an operator who operates the operating section, and second monitor means disposed around hands of the operator, and information from the operating section is sent to the clinic of the video outputted by the second monitor means.

According to this aspect, even when accessing a plurality of clinics from one medical cockpit, it is possible to control each clinic without making mistake, and the operator can concentrate on the operation of a surgical site using the second monitor means disposed near hand of the operator.

### BRIEF DESCRIPTION OF THE DRAWINGS

FIG. 1 is a block diagram showing the entire structure of a medical cockpit system of the present invention;

FIG. 2 is a conception view showing a concrete structure of a clinic in the medical cockpit system of the invention;

FIG. 3 is a conception view showing a concrete structure of a medical cockpit in the medical cockpit system of the invention;

FIG. 4 is a block diagram of a medical cockpit system having a plurality of medical cockpits of the invention;

FIG. 5 is a conception view showing another monitor structure in a cockpit of the invention; and

FIG. 6 is a conception view for explaining a case in which predetermined motion of a doctor in the cockpit is utilized as a trigger signal.

### BEST MODE FOR CARRYING OUT THE INVENTION

An embodiment of the present invention will be explained based on the drawings below.

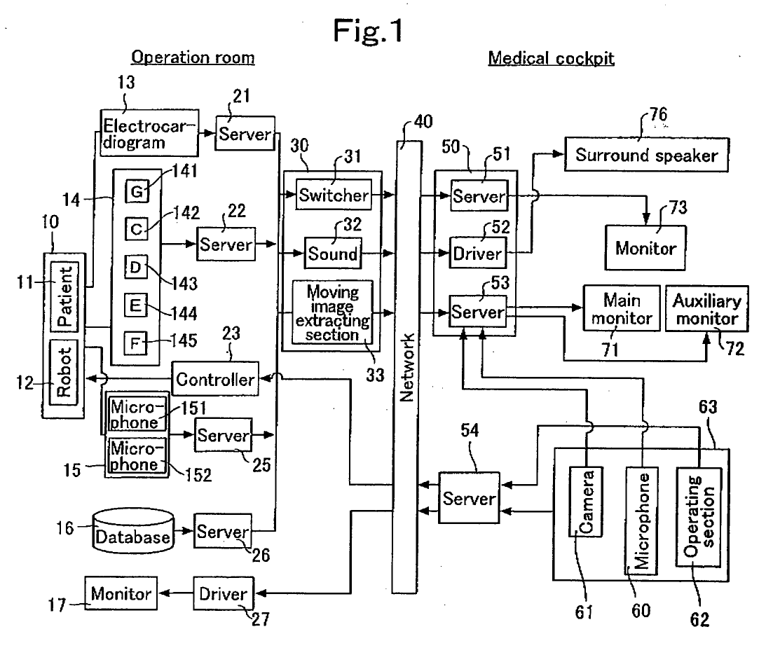

FIG. 1 is a block diagram showing the entire structure of a medical cockpit system of an embodiment. In an operation room, a patient 11 is lying on an operating table 10. An operation manipulator or a robot (robot, hereinafter) 12 is disposed as an operation tool near the operating table 10. The robot 12 carries out surgical operation motion in accordance with instructions of the controller 23.

In an operation room, a measurement information obtaining apparatus 13 such as electrocardiogram for measuring electrocardiogram information for obtaining an electrocardiogram of a patient is disposed. The information is accumulated in a measurement information server 21. In the operation room, there are a camera apparatus (operation room camera means) 141 for shooting a state of the operation room and camera apparatuses (body information camera means) 142, 143, 144 and 145 for shooting a body including an affected part of the patient 11.

Here, it is preferable that the camera apparatus 141 includes, in addition to the camera apparatus for shooting the state of the entire operation site (peripheral vision video) around an operating table 10, a camera apparatus for shooting a staff existing in the operation room, and a camera apparatus for shooting the various apparatuses and states in the operation room as camera apparatuses for shooting local video. As the camera apparatus 141 for shooting the peripheral vision video, an omnidirectional camera apparatus capable of shooting omnidirectional images is suitable. The plurality of camera apparatuses 142, 143, 144 and 145 disposed on the robot 12 shoot the affected part of the patient from various directions with respect to the patient. The number and positions of the camera apparatuses 142, 143, 144 and 145 appropriately are changed in accordance with the affected part or kinds of operation and disposed movably. The video information shot by the camera apparatus 141 is accumulated in the video information server 22.

A microphone 152 is disposed on the operating table 10 in the vicinity of the patient 11. The microphone 152 captures the operating sound of the affected part of the patient and breathing sound of the patient. A microphone 151 is attached to a breast of a staff such as a nurse who is involved in the operation to capture the voices talking and sound. The microphones 151 and 152 constitute a voice information obtaining apparatus 15 which captures sound which is related to the surgical operation in the operation room. The voice information in the operation room captured by the voice information obtaining apparatus 15 is accumulated in the voice information server 25. It is preferable that the voice information obtaining apparatus 15 includes position signal generating means. In this case, the camera apparatus 14 includes receiving means of the position signal and driving means which is driven by this signal. That is, the voice information obtaining apparatus 15 includes the position signal generating means, and the camera apparatus 14 includes the receiving means and the driving means. With this configuration, it is possible to shoot images while following a moving body. In this case, it is preferable that a position signal is sent to the video information server 22 together with the video information. By sending the position signal together with the video information in this manner, it is possible to move the local video on a monitor in the medical cockpit. As position detecting methods of a moving body such as a nurse in an operation room, there are a position measuring method using ultrasonic type, optical type or magnetic type transmitter receiver, a position measuring method using only a receiver (camera apparatus) which measures or recognizes images, and an autonomous navigation method which measures a position from a moving distance and a moving direction from an arbitrary original point. The position can be detected by using such methods alone or in combination. Based on the position detection information, local video is moved and displayed on a monitor in the medical cockpit. Also when a voice generating source moves, it is preferable that the stereophonic is realized in the medical cockpit based on the position detection information.

A database 16 accumulates data such as various physical data previously obtained by medical examination of a patient before the surgical operation, such as tomography image data of MRI, CT or echo around the affected part, blood data, characteristic data such as size, color and position of an organ, and data such as medical history and habit obtained by hearing with the patient. Further, data concerning medical information related to the surgical operation, medical procedure schedule (treatment schedule or operation schedule) is also accumulated in the database 16 if necessary. It is not necessary that the database 16 is in the operation room, and the database 16 may be taken out from a database which is collectively managed in a hospital. Desired data which is obtained from the database 16 is accumulated in a database server 26.

The information accumulated in the measurement information server 21, the video information server 22, the voice information server 25 and the database server 26 is integrated by an integration server 30 and is sent through a network 40 to the medical cockpit where a doctor (operator) who performs the surgical operation (simply, “doctor”, hereinafter) exists. The integration server 30 includes a switcher 31 which switches at arbitrary intervals and outputs information from the measurement information obtaining apparatus 13 supplied from the measurement information server 21, video information from the camera apparatuses 141, 142, 143, 144 and 145 supplied from the video information server 22, information from the database 16 supplied from the database server 26, and video information from the camera apparatuses 141, 142, 143, 144 and 145. The integration server 30 also includes a voice information extracting section 32 which selects voice information from the voice information server 25, and a moving image extracting section 33 which extracts, as real time moving image, the video information supplied from the measurement information server 21, the video information server 22 and the database server 26.

The operation room is provided with a driver 27 and a monitor 17 which reproduce video information corresponding to motion of the doctor in the medical cockpit and voice talking and sound of the doctor. The operation room is also provided with a monitor 17.

In the medical cockpit, an integration server 50 receives information from the integration server 30 sent through the network 40. The integration server 50 comprises an image server 51 which sequentially accumulates image information which is switched and obtained by the switcher 31, a voice information driver 52 which accumulates voice information from the voice information extracting section 32 and develops the same in a multi-channel manner, and a moving image server 53 which accumulates moving image information from the moving image extracting section 33.

A freeze-frame picture from the image server 51 is displayed on a large-screen monitor (first monitor means) 73. The monitor 73 includes a large screen for displaying the entire operation room (peripheral vision), and a plurality of small screens (catalog screens) located or inset at predetermined positions of the large screen. The small screens display measurement information supplied from the measurement information server 21, video information of the affected part of the patient from the camera apparatuses 142, 143, 144 and 145 as local video supplied from the video information server 22, and video information supplied from the database server 26. The inset small screen is disposed at a location where the doctor in a normal operation room sees the information. Therefore, the doctor can see the image disposition in the monitor 73 as if the doctor is in the operating position in the actual operation room although the doctor is in the remote location away from the operation room. The inset small screen may be disposed at the video position in the video which is outputted by the peripheral vision video where the video contents are outputted in the small screen. By disposing the small screen in relation to the video contents of the peripheral vision video in this manner, it is possible to exactly discriminate the video of the necessary small screen. Since the video outputted on the small screen is the intermittently received video information, it is not always necessary that the number of transmitting lines is the same as the number of camera apparatuses, and it becomes easier to handle more local videos.

The voice information in the operation room developed into a multi-channel signal by the voice information driver 52 is supplied to a surround speaker 76, and is reproduced in the medical cockpit in a multi-channel manner.

Among the moving image information from the moving image server 53, the moving image information of the affected part around a cutting edge of a knife is always outputted on a main monitor (second monitor means) 71. On the other hand, among the moving image information from the moving image server 53, moving image information selected by the doctor and an enlarged image are displayed on an auxiliary monitor (second monitor means) 72.

The main monitor 71 and the auxiliary monitor 72 are disposed around hands of the doctor. The doctor carries out the surgical operation by operating an operating section 62 of an operating position 63 while seeing the images on the main monitor 71 and the auxiliary monitor 72. In the operating position 63, a microphone 60 capturing the voice talking and sound of the doctor is disposed, and moving image information or enlarged image is to be outputted on the auxiliary monitor 72 is selected in accordance with the voice talking or sound of the doctor. A camera apparatus 61 for shooting the motion of the doctor is disposed in place in the medical cockpit. When the doctor carries out predetermined motion, moving image information or enlarged image to be outputted on the auxiliary monitor 72 may be selected. When the predetermined motion of the doctor is used as a trigger signal, image from the camera apparatus 61 which shoots the motion of the doctor may be recognized or a position or motion of a trigger generating apparatus attached to a body of the doctor may be detected. However, when the predetermined motion of the doctor is used as the trigger signal, it is preferable that the voice recognition is also used. As one example of the predetermined motion of the doctor, a foot switch may be used. Two or more main monitors 71 and the auxiliary monitors 72 may be provided. In the main monitor 71, it is preferable that the video is not changed over during the operation of the medical procedure system.

A signal from the operating section 62 which is operated by the doctor is supplied to a server 54, and is supplied to the controller 23 in the operation room through the network 40. The controller 23 drives the robot 12 in real time in accordance with the motion of the operating section 62, and carried out predetermined motion of the surgical operation.

The voice talking of the doctor captured by the microphone 60 and the state of the doctor shot by the camera apparatus 61 are accumulated in the server 54, they are supplied to the driver 27 in the operation room through the network 40 and are outputted on the monitor 17. Therefore, the staff in the operation room can see the state of the doctor, and can receive the instructions from the doctor.

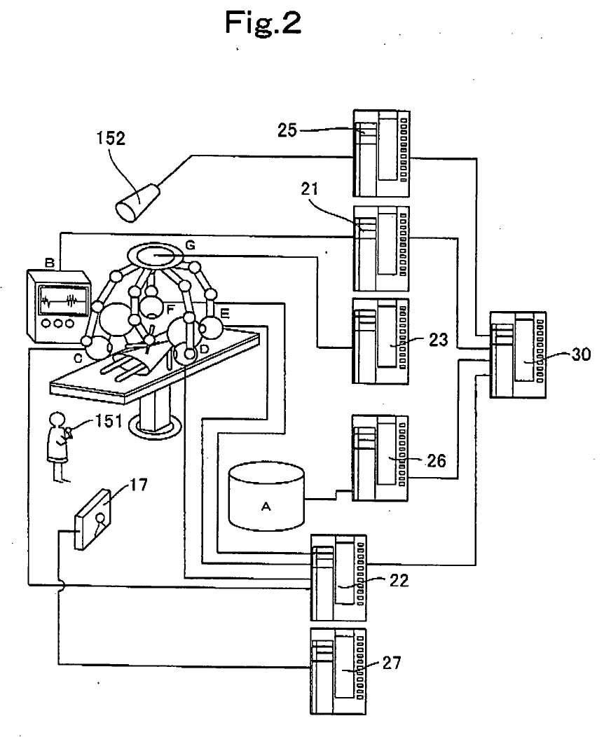

FIG. 2 is a conception view showing a concrete structure of the operation room of the remote surgical operation system shown in FIG. 1. The microphone 151 is attached to a breast of a staff who is involved in the operation, and the microphone 151 captures the voices talking of the staff and sends the same to a server 25. The staff moves in the operation room and thus, the position of the voice talking also moves. Therefore, it is preferable that the microphone 151 is of a wireless type. The breathing sound of the patient and the operation sound of the affected part are captured by the microphone 152 and sent to the voice information server 25. The microphone 152 may be of a wire type or a wireless type.

A state of the entire operation site around the operating table 10 is shot by the camera apparatus 141, and its moving image signal G is accumulated in the video information server 22. The camera apparatuses 142, 143, 144 and 145 shoot the affected part of the patient 11 from various directions of the patient, and moving image data C, D, E and F is accumulated in the video information server 22.

The measurement information obtaining apparatus 13 extracts electrocardiogram of the patient, and measurement information B comprising sound information which is in association with the waveform diagram of the electrocardiogram and waveform thereof is accumulated in the measurement information server 21. The measurement information obtaining apparatus 13 may be apparatus other then the electrocardiogram.

The information A from the database 16 is supplied to the database server 26. It is not always necessary that the database 16 and the database server 26 are disposed in the operation room as described above.

Outputs from the voice information server 25, the video information server 22, the measurement information server 21 and the database server 26 are integrated by the integration server 30 and are sent to a medical cockpit at a remote location where the doctor exists or a remote location where a doctor of diagnosis exists.

The operation action of the doctor sent from the medical cockpit through the network 40 is sent to a driver 23 in the operation room, and the driver 23 drives a knife operating section 121 of the robot 12 to carry out the surgical operation. The motion and voice of the doctor are supplied to the driver 27, and reproduced by the monitor 17 in the operation room.

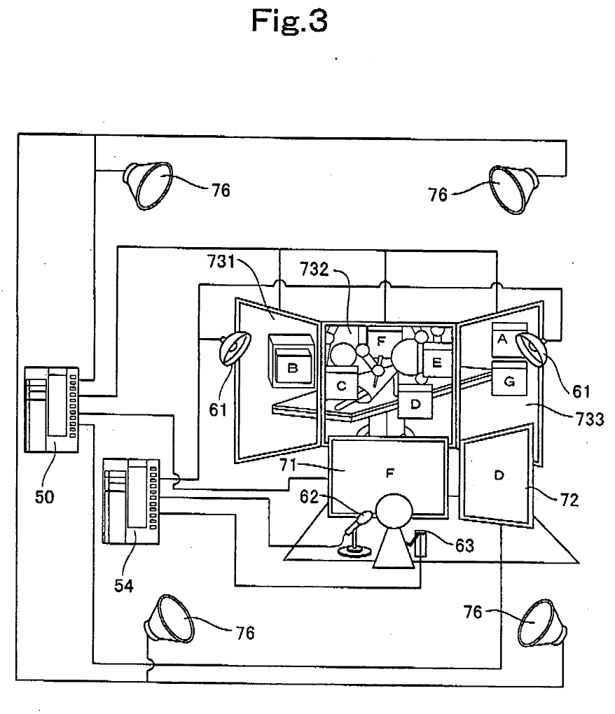

FIG. 3 is a conception view showing a concrete structure of the operation room of the medical cockpit shown in FIG. 1. It is most preferable that the monitor 73 shown in FIG. 1 is a cylindrical immersive display having a horizontal viewing angle in a range of 120° to 330°, a projection type or transmission type curved screen at a position of the doctor who operates the operating section. Alternatively, a plurality of monitors, e.g., three monitors 731, 732 and 733 may be disposed in a side-by-side manner. If the center viewing angle of a person is taken into account, the viewing angle is preferably 120° or greater, and in order to cover a peripheral viewing angle of a person, the viewing angle is preferably 270° or greater, and if motion of a head of the operator is taken into account, the viewing angle of about 330° is more preferable. For example, three wide-angle projection displays of 60 inches may be disposed at an angle of 30° therebetween. When three monitors are used, it is preferable that the center field of view of the center monitor 732 has 120(, and the viewing angle of the entire monitors including the both side monitors 731 and 733 is 270° or greater. It is preferable that two more monitors are continuously disposed on both sides to further increase the viewing angle. On the monitors 731, 732 and 733 disposed in this side-by-side manner, the state of the entire operation site shot by the camera apparatus 141 is largely and continuously outputted.

As shown in the drawing, an image A of the database 16 and an image G which is the other local video are inset in the one (right side) monitor 733. Images C, D, E and F of the affected part of the patient are inset in the center monitor 732, and an electrocardiogram image B is inset in the other (left side) monitor 731. Beep sound corresponding to the waveform of the electrocardiogram is also reproduced at the same time from the inset position of the electrocardiogram image B from the position of the doctor. These inset images are sequentially outputted as intermittent images which are obtained by switching, by the integration server 50, the moving image sent through the network 40. Therefore, it is possible to simultaneously obtain the all kinds of images required for the entire operation site in the operation room and operation by seeing the monitors 731, 732 and 733.

The main monitor 71 and the auxiliary monitor 72 are disposed in the vicinity of hands of the doctor. The moving image of image selected from the images A to G which are inset in the monitors 731, 732 and 733 are enlarged and displayed on the main monitor 71 and the auxiliary monitor 72. In the example shown in FIG. 3, a moving image corresponding to the image F is displayed on the main monitor 71 in the enlarged manner, and a moving image corresponding to the image D is displayed on the auxiliary monitor 72 in the enlarged manner. When images are displayed on the main monitor 71 and the auxiliary monitor 72, they are displayed as real time video.

The sound in the operation room sent from the voice information server 25 in the operation room is reproduced in the multi-channel manner by the plurality of surround speakers 76. When voice is generated from the peripheral vision video outputted on the monitor 73 or video outputted by local videos A, B, C, D, E, F and G using the surround speakers 76, it is preferable that the voice is reproduced as stereophonic so that voice can be heard in correspondence with the position of the video. It is preferable that the voice is reproduced as stereophonic so that the voice can be heard at the position of the doctor in the medical cockpit in a state in which the direction and the distance of the voice heard at the original position of the doctor in the operation room are the same.

In the medical cockpit, there are disposed the microphone 60 for capturing the voice talking of the plurality of camera apparatuses 61 which shoot the doctor, and the operating section 62 for operating the robot 12 in the operation room. Signals from the camera apparatus 61, the microphone 60 and the operating section 62 are supplied to the server 54, and sent to the operation room through the network 40.

The operation of the remote surgical operation system of the present invention will be explained with reference to FIGS. 1 to 3. In the following explanation, the monitor 73 of the medical cockpit comprises the three monitors 731, 732 and 733 as shown in FIG. 3.

The camera apparatuses 141, 142, 143, 144 and 145 for shooting the operation site and the patient are set at predetermined positions. An electrode of the measurement information obtaining apparatus 13 is mounted on the patient. The microphone 151 is attached to a breast of a staff who is involving in the surgical operation. The microphone 152 is disposed near the patient 11. Data such as physical tomography image of the patient 11 previously accumulated in the database 16 is sent to the database server 26. The monitor 17 is turned ON, and video of the doctor sent from the medical cockpit at a remote location is outputted on the monitor 17.

Signals from the camera apparatuses 141, 142, 143, 144 and 145 are sent to the video information server 22. Video and sound from the measurement information obtaining apparatus 13 are sent to the measurement information server 21. Voice from the microphones 151 and 152 is sent to the voice information server 25. Data from the database 16 is sent to the database server 26. Data of the servers 21, 22, 25 and 26 is integrated by the integration server 30 and sent to the medical cockpit through the network 40. In the integration server 30, the switcher 31 switches at arbitrary intervals and outputs information from the measurement information obtaining apparatus 13 supplied from the measurement information server 21, video information from the camera apparatuses 141, 142, 143, 144 and 145 supplied from the video information server 22, and information from the database 16 supplied from the database server 26, and the voice information extracting section 32 selects voice information from the voice information server 25, and the moving image extracting section 33 extracts and sends, as moving image, the video information supplied from the measurement information server 21, the video information server 22 and the database server 26. The data integrated by the integration server 30 may be sent as it is without through the switcher 31, the voice information extracting section 32 and the moving image extracting section 33, and the medical cockpit may be provided with the switcher 31, the voice information extracting section 32 and the moving image extracting section 33.

In the medical cockpit, information from the integration server 30 sent through the network 40 is received by the integration server 50. Of the output from the image server 51 which accumulates the freeze-frame picture information switched and obtained by the switcher 31, the integration server 50 insets the information from the measurement information obtaining apparatus 13 in the monitor 731 at the position shown with B, and reproduces the waveform diagram and sound. Similarly, of the output from the image server 51, freeze-frame pictures which are videos of the affected part of the patient sent from the camera apparatuses 142, 143, 144 and 145 are inset and reproduced in the monitor 732 at positions shown with C, D, E and F. Of the output from the image server 51, a freeze-frame picture of the entire operation site from the camera apparatus 141 and data image from the database 16 are inset and reproduced in the monitor 733 at positions shown with G and A. These inset positions match with dispositions of various apparatuses in a normal operation room so that the doctor can see the image and hear the sound in the same environment where the doctor usually sees and hears in the operation room.

The voice information in the operation room developed into a multi-channel signal by the voice information driver 52 of the integration server 50 is supplied to the surround speaker 76, reproduced in the medical cockpit room in the multi-channel manner, and is outputted in a state in which a voice environment in the medical cockpit matches with a voice environment of the operation room. Therefore, the doctor can hear various voices generating in the operation room such as voice of the staff, breathing sound from a respirators in the same environment as that when the doctor stands at the operating position in the operation room.

Of the moving image information from the moving image server 53 of the integration server 50, enlarged moving image information of the affected part around the cutting edge of the knife is always outputted on the main monitor 71. Of the moving image information from the moving image server 53, the enlarged moving image information or the enlarged image selected by the doctor is displayed on the auxiliary monitor 72. The doctor carries out the surgical operation by operating the operating section 62 while seeing the enlarged image on the main monitor 71 and the enlarged moving image information or the enlarged image on the auxiliary monitor 72 which is necessary occasionally. The operation of the operating section 62 is supplied to a controller in the operation room from the server 54 through the network 40, and the controller 23 operates the knife operating section 121 of the robot 12 to carry out the surgical operation. At that time, if the body tomography image is taken out from the database 16 and the tomography image is superposed on, in a transparent manner, or is displayed adjacent to an image of the affected part outputted on the main monitor 71, further precise surgical operation can be carried out. The state of the doctor is shot by the camera apparatus 61, his or her voice is captured by the microphone 60 and is sent to the driver in the operation room from the server 54 through the network 40, and is reproduced on the monitor 17. Therefore, when doctor desires to give instructions to the staff, if the doctor instructs the staff through the microphone 60, the staff can receive the instructions by seeing the monitor 17.

Usually, the doctor uses his or her both hands during the surgical operation. Thus, it is preferable that the doctor can select the enlarged moving image information or enlarged image to be reproduced on the auxiliary monitor 72 by giving the selection instructions through the microphone 60 or by giving the instructions by means of gesture when the camera apparatus catches a particular body motion of the doctor. For example, when it is necessary to enlarge and display the electrocardiogram on the auxiliary monitor 72, if the doctor says “electrocardiogram”, a server 53 recognizes the voice and receives the instructions to that effect, and selects the moving image information of the electrocardiogram and supplies the same to the auxiliary monitor 72. The images may be switched by operating a foot switch or pushing a selection switching button of course. As instructions by means of the body motion, motions of arm, hand or finger of the doctor, or motions of chin, nose, eyebrow, eye or mouth which are characteristic portions of a face may be recognized, the motion direction of the body portion may be discriminated, and the discriminated result may be used as a trigger signal.

The motion of the operating section 62 and the motion of the knife operating section 121 of the robot 12 are associated with each other. If the motion amount when the motion of the operating section 62 is converted into the motion of the knife operating section 121 by signal processing of a transmitting system is scaled down, it is possible to realize fine motion of the knife operating section 121 or to eliminate shake motion of hand of the operating section 62. For example, if the motion of the operating section 62 is scaled down by 1/100 and the knife operating section 121 is moved, the knife operating section 121 moves by 1 mm when the operating section 62 is moved by 10 cm, and it is possible to easily carry out a fine incision operation or the like. A shake of hand of about 5 mm of the operating section 62 is converted into motion of 0.05 mm of the knife operating section 121, and this amount is negligible as a moving amount at the time of surgical operation and thus, the shake of hand is eliminated. The scale change can freely be carried out by using a method of changing the setting of the operation knob of the server 54, instructing through the microphone 60 or the like.

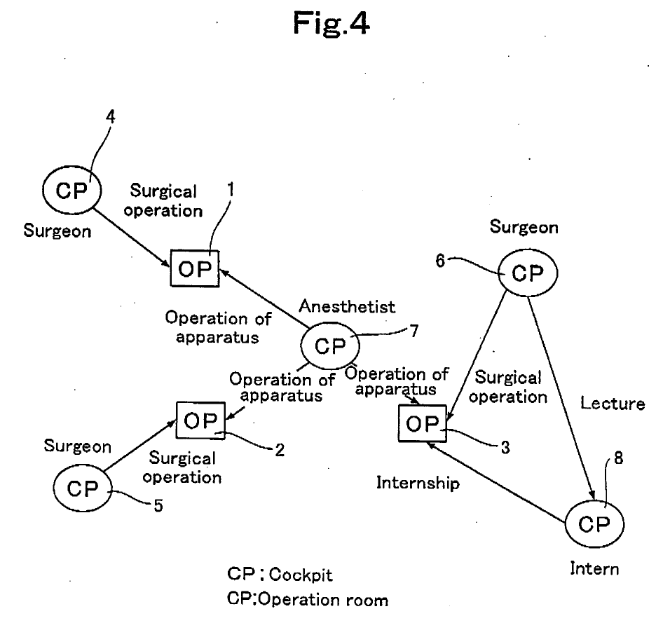

In the above embodiment, the information is sent and received by two points, i.e., between the operation room and the medical cockpit. It is also possible to send and receive information between different medical cockpits as shown in FIG. 4.

FIG. 4 is a block diagram of the medical cockpit system having a plurality of medical cockpits.

As shown in FIG. 4, when a plurality of clinics 1, 2 and 3 exist, the clinics 1, 2 and 3 carry out medical procedure such as surgical operation in accordance with instructions from medical cockpits 4, 5 and 6, respectively. From the cockpit 7, advice from an anesthetist is given to the clinics 1, 2 and 3. Since a cockpit 8 for an intern is connected between the clinic 3 and the medical cockpit 6, the intern can see the clinic 3 for study while receiving lecture of a doctor existing in the medical cockpit 6.

If the structure of the medical cockpit except the operating section 62 is provided in another location, it is possible to carry out the remote surgical operation while sending and receiving information between three or more medical stations.

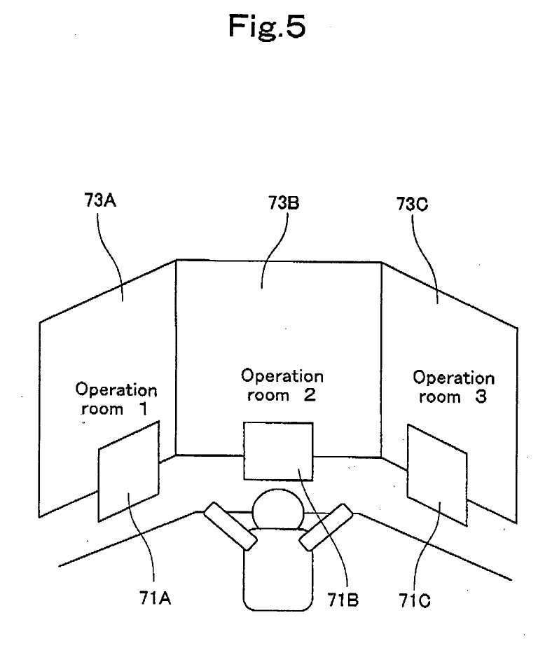

It is preferable that a monitor structure of the cockpit 7 used by the anesthetist as shown in FIG. 4 is as shown in FIG. 5.

FIG. 5 is a conception view showing another monitor structure in the cockpit. Of a concrete structure of the medical cockpit shown in FIG. 3, FIG. 5 shows only the monitor structure.

As shown in FIG. 5, in the cockpit, there are a plurality of large screen monitors (first monitor means) 73A, 73B and 73C, and a plurality of main monitors (second monitor means) 71A, 71B and 71C. The large screen monitors (first monitor means) 73A, 73B and 73C may display the entire operation rooms (peripheral visions), respectively, or the peripheral vision videos of the respective operation rooms may be selectively switched and displayed. It is preferable that the main monitors (second monitor means) 71A, 71B and 71C display local videos of the respective operation rooms, especially moving image information of the affected part. When one medical cockpit is connected to a plurality of clinics, it is necessary that the clinics can be controlled in a time division manner, and information from the operating section is sent to the corresponding clinic precisely. Therefore, it is preferable that the information from the operating section, the information from the cockpit camera means and the information from the cockpit voice information obtaining means at least concerning the instructions are selectively time-divided and sent to the respective clinics. It is preferable that the information concerning the instructions of the operator is selected such that the information is sent to the clinic of the video outputted by the second monitor means disposed near the hands of the operator.

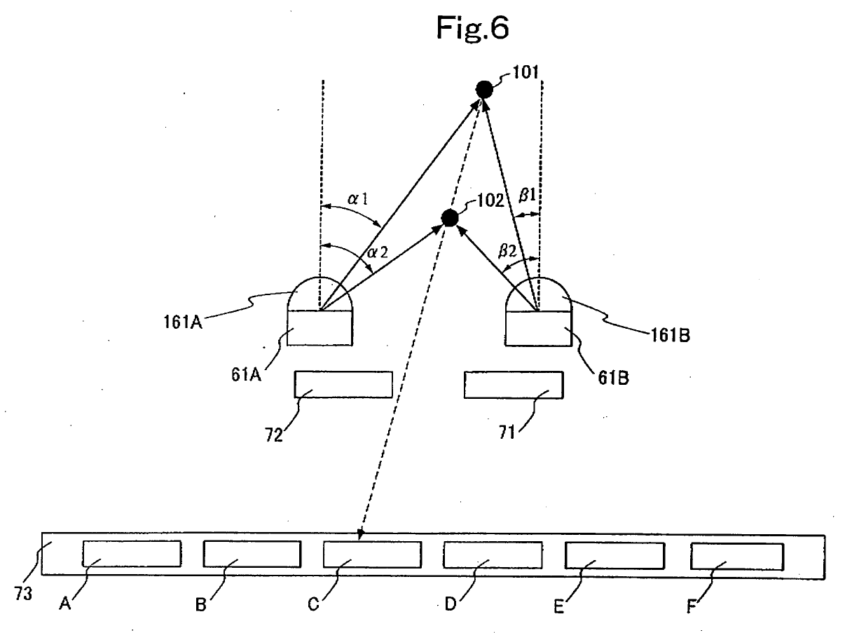

FIG. 6 is a conception view for explaining a case in which predetermined motion of the doctor in the cockpit is utilized as a trigger signal.

It is possible to recognize arm, hand or finger, or chin, nose, eyebrow, eye or mouth, of the doctor, which are characteristic portions of a face, but here a case in which the finger is recognized will be explained. Although it is possible to recognize the finger itself, if an LED is attached to the finger, it becomes easier to detect the detecting section.

FIG. 6 shows a state in which two shooting apparatuses 61A and 61B detect a light source position of the LED. The two shooting apparatuses 61A and 61B are disposed at positions closer to the doctor than the second monitor means 71 and 72. The shooting apparatuses 61A and 61B are respectively provided with fish-eye lenses 161A and 161B.

In FIG. 6, catalog screens A, B, C, D, E and F are small screens outputted in the first monitor means 73 which has been explained with reference to FIG. 3. A light source 101 shows a measuring position at first measuring time, and a light source 102 shows a measuring position at second measuring time. In this embodiment, the explanation is made on a two dimensional basis as viewed from above but in an actual case, measurement and detection are carried out three dimensionally. In this embodiment, two measuring positions of the light source 101 and the light source 102 are used, but three or more measuring positions may be detected, and measurement may be carried out using predetermined data from these detection data.

Position data of the shooting apparatuses 61A and 61B and catalog screens A, B, C, D, E and F is previously registered in the database.

In this structure, first, a position of the light source 101 is measured. In this measurement, an angle α1 of the light source 101 is measured from an optical axis of the fish-eye lens 161A of the shooting apparatus 61A, and an angle β1 of the light source 101 is measured from the optical axis of the fish-eye lens 161B of the shooting apparatus 61B. The position information of the light source 101 is calculated from the position data of the shooting apparatuses 61A and 61B and angle data thereof. Next, the position of the light source 102 is measured. In this measurement, an angle (2 of the light source 102 is measured from an optical axis of the fish-eye lens 161A of the shooting apparatus 61A, and an angle (2 of the light source 102 is measured from the optical axis of the fish-eye lens 161B of the shooting apparatus 61B. The position information of the light source 102 is calculated from the position data of the shooting apparatuses 61A and 61B and angle data thereof. A moving direction from the light source 101 to the light source 102 is calculated from the position information of the light source 101 and the position information of the light source 102, and a catalog screen C which is on the extension of the moving direction is estimated from the moving direction and the position information of the light source 101 or the light source 102. It is determined that this estimated catalog screen C is selected, and the video of the catalog screen C is displayed on the auxiliary monitor 72.

In this embodiment, the catalog screens A to F of the instruction direction are calculated using the plurality of light sources 101 and 102. Alternatively, position data may previously be registered while defining the doctor's position as a reference position, and the catalog screens A to F in the instruction direction may be calculated from the reference position data and the position data of the light sources. The measuring position of the light source may be specified by another trigger signal such as voice of the doctor or a foot switch. It is also effective that instruction information measured before the actual surgical operation or instruction information measured during the actual surgical operation is stored, a position relation between measurement data from the light sources 101 and 102 and the catalog screens A to F is stored for each doctor, and this relation is used as correction information. If such correction information is used, deviation from the actual position relation in the instruction direction which is generated from difference between the doctors can be corrected, and the catalog screens A to F which are desired by the doctor can be selected.

The explanation of this embodiment is based on the surgical operation, but the present invention can also be applied to a remote medical procedure system in medical procedure such as examination, inspection, diagnosis, treatment and other proceeding. Therefore, the operation room may be a clinic, the operation table may be a medical procedure table, the operation manipulators or the robot as the operating means may be medical procedure means comprising the medical procedure manipulator or the robot.

Although the various servers are used and data is accumulated in the servers in the embodiment, the server need not always accumulate data, and may have a function for controlling the sending and receiving operations of data.

Although the robot moves based on the instruction information from the medical cockpit in the embodiment, the robot may have autonomous function such as a danger-avoiding function. Alternatively, a basic motion program which is previously registered in a database and which causes motion based on various actual detection data may be prepared, and the robot may move in accordance with the basic motion program, and the operator may instruct the selection of the basic motion program or switching to a manual operation.

Although the main monitor explained in the above embodiment is the two dimension video, a stereoscopic video or a three dimension video are more preferable.

The clinic voice information obtaining means explained in the above embodiment may be microphones provided on the plurality of camera apparatuses 142, 143, 144 and 145 disposed on the robot 12. If shooting means and a microphone are provided on an arm tip end of the robot 12, positions can be recognized precisely using data which is used for controlling the robot.

Although the medical procedure system is for a human in the above embodiment, the medical procedure system can be used as it is even for an animal.

If the video and voice data concerning the medical procedure accumulated in the server are reproduced later, they can be used as simulation internship data.

Although the fish-eye lens is used in the embodiment shown in FIG. 6, a wide-angle lens or a compound eye lens may be used. Alternatively, it is unnecessary to use a lens, and an artificial retina chip or a shooting apparatus comprising general shooting device may be used.

In the embodiment shown in FIG. 6, the two shooting apparatuses 61A and 61B are disposed closer to the doctor than the second monitor means 71 and 72. Alternatively, the shooting apparatuses 61A and 61B may be disposed at other positions, or three or more shooting apparatuses may be provided.

It is preferable that the instructions by means of a body motion of the doctor are displayed using display means in the operation room.

In the embodiment, the catalog screens A to F are selected by the instructions by means of the body motion of the doctor. Alternatively, a specific affected part position of diagnosis or treatment can be selected by instructions by means of the body motion of the doctor. When the specific affected part position is selected, it is preferable that the selection contents are displayed by means of the display means on the side of the operation room.

### INDUSTRIAL APPLICABILITY

According to the medical cockpit system of the present invention, a doctor existing at a remote location can carry out medical procedure for a patient as if the doctor is directly carrying out the medical procedure in a clinic. Therefore, even in a region or area where the number of doctors is small such as an underpopulated region, it is possible to receive medical procedure by a specialist physician only by carrying out infrastructural project of the clinic.

Since it is possible to store and accumulate images and voice data during the medical procedure, it is possible to contribute to the medical technique improvement.

Many effects can be obtained by the present invention. For example, if the medical cockpit system of the present invention is realized, it is possible to present such universal medical service that high medical care can be received anywhere like an underpopulated region or a vacationland. It is possible to reduce physical and financial burden on a patient caused by locomotion, to allow medical technique to become widespread, and to receive medical care using mother tongue even at abroad. Since it is possible to receive treatment of a physician in charge, confidence between the doctor and the patient can be utilized. For example, night time medical procedure can currently be received at day time place.

1. A medical cockpit system in which a clinic and a medical cockpit are connected to each other through a network, said clinic comprises measurement information obtaining means for obtaining measurement information such as electrocardiogram information of a patient, clinic camera means for shooting a state in said clinic including at least a medical procedure table, clinic voice information obtaining means for obtaining voice in said clinic, physical information camera means for shooting a body including an affected part of said patient, and medical procedure means for carrying out medical procedure for said patient by remote control, and said medical cockpit comprises monitor means for outputting video from said measurement information obtaining means, said clinic camera means and said physical information camera means, voice reproducing means for reproducing voice information from said clinic voice information obtaining means, and an operating section for remote controlling said medical procedure means, wherein said monitor means comprises first monitor means disposed at a predetermined distance from an operator who operates said operating section, and second monitor means disposed around hands of said operator, said first monitor means outputs a peripheral vision video in said clinic shot by said clinic video means and outputs a plurality of local videos in said clinic as catalog screens such that said local videos are partially overlapped on said peripheral vision video, and said second monitor means outputs measurement information obtained by said measurement information obtaining means or video from said physical information camera means.

2. The medical cockpit system according to claim 1, further comprising database means in which previously obtained physical data of said patient or data peculiar to said patient is accumulated, wherein data accumulated in said database means is sent together with said information in said clinic.

3. The medical cockpit system according to claim 1, further comprising sending means which sends said information sent from said clinic not only to said medical cockpit but also to another cockpit.

4. The medical cockpit system according to claim 1, wherein said physical information camera means comprises a plurality of camera apparatuses which shoot said affected part from a plurality of directions.

5. The medical cockpit system according to claim 1, wherein said first monitor means is disposed such that a viewing angle in said horizontal direction at a position of said operator who operates said operating section is in a range of 120° to 330°.

6. The medical cockpit system according to claim 1, wherein said second monitor means comprises a main monitor and an auxiliary monitor, said main monitor outputs said measurement information obtained by said measurement information obtaining means or said video from said physical information camera means, and said auxiliary monitor selectively switches and outputs catalog screens outputted on said first monitor means.

7. The medical cockpit system according to claim 1, wherein said second monitor means comprises a plurality of monitors, at least one of said monitors does not switch videos during operation of said medical procedure system.

8. The medical cockpit system according to claim 6 or 7, wherein said auxiliary monitor switches said catalog screens by detecting voice, motion and countenance of said operator.

9. The medical cockpit system according to claim 6 or 7, wherein said auxiliary monitor switches said catalog screens using a foot switch.

10. The medical cockpit system according to claim 1, wherein said medical cockpit comprises cockpit camera means for shooting motion or countenance of said operator, and cockpit voice information obtaining means for obtaining voice of said operator, video shot by said cockpit camera means and voice obtained by said cockpit voice information obtaining means are sent to said clinic through said network.

11. The medical cockpit system according to claim 1, wherein said clinic voice information obtaining means is a microphone held by a staff in said clinic.

12. The medical cockpit system according to claim 1, wherein said clinic voice information obtaining means is a microphone which captures sound in said vicinity of said affected part of said patient and breathing sound of said patient.

13. The medical cockpit system according to claim 1, wherein said catalog screen in said first monitor means is disposed in a video position on which video contents outputted on said catalog screen is outputted, of videos outputted by said peripheral vision video.

14. The medical cockpit system according to claim 1, wherein said local video outputted on said catalog screen of said first monitor means is video information which is intermittently received.

15. The medical cockpit system according to claim 1, wherein said voice reproducing means is a multi-speaker system, said voice is reproduced in a stereophonic manner such that said voice can be heard at a position of said operator in said medical cockpit in said same direction or at said same distance as that of said voice heard at an original position of said operator in said clinic.

16. The medical cockpit system according to claim 1, wherein said voice reproducing means is a multi-speaker system, when voice is generated from a peripheral vision video outputted by said monitor means or from video outputted by said local video, said voice is reproduced in a stereophonic manner so that said voice can be heard in correspondence with said position of said video.

17. The medical cockpit system according to claim 2, wherein data stored in said database means is superposed on video of said physical information camera means in a transparent manner, or displayed adjacent to said video of said physical information camera means.

18. A clinic connected to a medical cockpit through a network, comprising measurement information obtaining means for obtaining measurement information such as electrocardiogram information of a patient, clinic camera means for shooting a state in said clinic including at least a medical procedure table, clinic voice information obtaining means for obtaining voice in said clinic, physical information camera means for shooting a body including an affected part of said patient, and medical procedure means for carrying out medical procedure for said patient by remote control, and a monitor for reproducing video and voice of said operator, wherein information from said measurement information obtaining means, said clinic camera means, said clinic voice information obtaining means and said physical information camera means is sent to said medical cockpit, video and voice information of said operator from said medical cockpit is received, and said medical procedure means is allowed to be operated based on said information from said medical cockpit.

19. A medical cockpit connected to a clinic through a network, comprising monitor means for outputting video of said clinic, voice reproducing means for reproducing voice in said clinic, an operating section for remote controlling medical procedure means of said clinic, cockpit camera means for shooting motion and countenance of an operator, and cockpit voice information obtaining means for obtaining voice of said operator, wherein video outputted by said monitor means and voice reproduced by said voice reproducing means are received from said clinic, and information from said operating section, said cockpit camera means and said cockpit voice information obtaining means is sent to said clinic.

20. The medical cockpit according to claim 19, wherein said monitor means comprises first monitor means disposed at a predetermined distance from an operator who operates said operating section, and second monitor means disposed around hands of said operator, said first monitor means outputs a peripheral vision video in said clinic shot by said clinic video means and outputs a plurality of local videos in said clinic as catalog screens such that said local videos are partially overlapped on said peripheral vision video.

21. The medical cockpit according to claim 20, wherein said catalog screen in said first monitor means is disposed in a video position on which video contents outputted on said catalog screen is outputted, of videos outputted by said peripheral vision video.

22. The medical cockpit according to claim 20, wherein said second monitor means comprises a main monitor and an auxiliary monitor, said main monitor outputs measurement information obtained by said measurement information obtaining means or video from said physical information camera means.

23. The medical cockpit according to claim 20, wherein said second monitor means comprises a main monitor and an auxiliary monitor, said auxiliary monitor selectively switches and outputs catalog screens outputted on said first monitor means.

24. The medical cockpit according to claim 19, wherein said medical cockpit is connected to a plurality of clinics, said monitor means comprises first monitor means disposed at a predetermined distance from an operator who operates said operating section, and second monitor means disposed around hands of said operator, and information from said operating section is sent to said clinic of said video outputted by said second monitor means.
